SEQUENCE LISTING (51 sequences)

SEQ ID NO: 1 - DNA, 1566 bp, Glycine max
atgttgctggaacttgcacttggtttgtttgtgttagctttgtttctgcacttgcgtcccacaccaagtgcaaaatcaaaagcacttcgccacctcccaaaccctccaagcccaaagcctcgtcttcccttcattggccaccttcacctcttaaaagataaacttctccactatgcactcatcgatctctccaaaaagcatggccccttattctctctctccttcggctccatgccaaccgtcgttgcctccacccctgagttgttcaagctcttcctccaaacccacgaggcaacttccttcaacacaaggttccaaacctctgccataagacgcctcacttacgacaactctgtggccatggttccattcggaccttactggaagttcgtgaggaagctcatcatgaacgaccttctcaacgccaccaccgtcaacaagctcaggcctttgaggacccaacagatccgcaagttccttagggttatggcccaaagcgcagaggcccagaagccccttgacgtcaccgaggagcttctcaaatggaccaacagcaccatctccatgatgatgctcggcgaggctgaggagatcagagacatcgctcgcgaggttcttaagatcttcggcgaatacagcctcactgacttcatctggcctttgaagtatctcaaggttggaaagtatgagaagaggattgatgacatcttgaacaagttcgaccctgtcgttgaaagggtcatcaagaagcgccgtgagatcgtcagaaggagaaagaacggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaa

SEQ ID NO: 2 - DNA, 1566 bp, Glycine max
atgttgcttgaacttgcacttggtttattggttttggctctgtttctgcacttgcgtcccacacccactgcaaaatcaaaagcacttcgccatctcccaaacccaccaagcccaaagcctcgtcttcccttcataggacaccttcatctcttaaaagacaaacttctccactacgcactcatcgacctctccaaaaaacatggtcccttattctctctctactttggctccatgccaaccgttgttgcctccacaccagaattgttcaagctcttcctccaaacgcacgaggcaacttccttcaacacaaggttccaaacctcagccataagacgcctcacctatgatagctcagtggccatggttcccttcggaccttactggaagttcgtgaggaagctcatcatgaacgaccttctcaacgccaccactgtaaacaagttgaggcctttgaggacccaacagatccgcaagttccttagggttatggcccaaggcgcagaggcacagaagccccttgacttgaccgaggagcttctgaaatggaccaacagcaccatctccatgatgatgctcggcgaggctgaggagatcagagacatcgctcgcgaggttcttaagatctttggcgaatacagcctcactgacttcatctggccattgaagcatctcaaggttggaaagtatgagaagaggatcgacgacatcttgaacaagttcgaccctgtcgttgaaagggtcatcaagaagcgccgtgagatcgtgaggaggagaaagaacggagaggttgttgagggtgaggtcagcggggttttccttgacactttgcttgaattcgctgaggatgagaccatggagatcaaaatcaccaaggaccacatcaagggtcttgttgtcgactttttctcggcaggaacagactccacagcggtggcaacagagtgggcattggcagaactcatcaacaatcctaaggtgttggaaaaggctcgtgaggaggtctacagtgttgtgggaaaggacagacttgtggacgaagttgacactcaaaaccttccttacattagagcaatcgtgaaggagacattccgcatgcacccgccactcccagtggtcaaaagaaagtgcacagaagagtgtgagattaatggatatgtgatcccagagggagcattgattctcttcaatgtatggcaagtaggaagagaccccaaatactgggacagaccatcggagttccgtcctgagaggttcctagagacaggggctgaaggggaagcagggcctcttgatcttaggggacaacattttcaacttctcccatttgggtctgggaggagaatgtgccctggagtcaatctggctacttcgggaatggcaacacttcttgcatctcttattcagtgcttcgacttgcaagtgctgggtccacaaggacagatattgaagggtggtgacgccaaagttagcatggaagagagagccggcctcactgttccaagggcacatagtcttgtctgtgttccacttgcaaggatcggcgttgcatctaaactcctttcttaa

SEQ ID NO: 3 - DNA, 788 bp, Glycine max
ttaagaaaaaatactttatttacatgtataagcttaagattaaaaccctaattatatataagatagaattatctgttaactattatattataccatatcagttgtcacaacatgtgtttctggggttattgcctcttgagttcaattgcaacttgttaagcaaatcagccggcttaagacctaagcaacacaagcaagggctttaggtttcaaaaaaaggtttcaatttttttttaatattaatatatctaaaaaaattattgtaaaattatatttgaaaataagttttaattaaaatattataactaaccgttaatctttttattggtactataaataataatcaatgagcaacaattcttcaccgacatcatatctttgttttaaaaaaataataattttaataaattatttgataaataaataaaagattttattcttaaatttattttaaatctctttgtgtccttgaaaagtccatgatacaggatgagatatttgactatttgactagaaacgtagtaggtgatatatggacatttcctggtttattttatattcttaaaaaataacaattcaatcgaatgtagttgccaaattttaataaataaataaaaagaagcattcatcgaattcttcgtcttttatgagtgtaaaacaaaacattgaattaggaacaattattatcacgttacttaaaataaaatatactaaaaccgttgaatgaaatcttcatatttgataagtgtaggtagacccacaacacaaacattgaatttgaataaatt

SEQ ID NO: 4 - DNA, 1142 bp, Glycine max
gcgttttaaacttttatactgacattaacgtcattattttaacaggcaaagagaaccaaaacaaaatttgtaatatatcagtcaccaattacaattttttttattgaaaacctaaaatatatttgactctaatttttttgctgatgcaagtctaaatttaattaaatttagacctaaaatatattgaaaacctaaatcattttttacaagataaaattaaattcttaactacctgttaagagataaaattaggtgtcaatcatattatgacatgctagttgtcaacacatttctttgactgaggcgcccgcctattgagctcaattgcaaataaaagaaacgtaatagatgatacatagatttttcgtgattcattatattttcgaatatagttggccgaataatctcatagtaaatttaaagaataaaaatatttgatgaaatttctaaaaccataattttaagtaggattaaattctgatgaaaaaccaatctcataatgaatctaaagaattaaaacttacaaaaatatttgatgaaaattctaattatgatgatactaaatcataataataaaaaaaaatatgatggcagcgatcaagacacgacaacatttcatttatttgaccgaggctgggtcacgaacgagtagcattccaaattgaaaatcgaaaaaaagaaaaggcgcagccgatcttagaccagagtatagtacatgtaccaaagaagtgtacctagtaggtgaagaagacaagttgaaaacaatcgccatcttaaaatcttttatttatttttttgactgaaaaaacaaacaactacagtactttttgagttttggaccattttgttaagtaaaatatggaaacatctgcatttaattcttgattcatttgtcactcttaatatattttcttgttccttaatgtcctctttaataggaagaataacaattttaacatgataaaaagggtggtaggcccacaagaattgaattgaatacaacaacaaaattccccataagtagagtatggtggagtagatgcttataaattggaatcttagaaggttgtttcctcaggccaagagcttggaaaattagcctcacaaaagcaaagatcaaacaaaccaaggacgagaacacg

SEQ ID NO: 5 - DNA, 17 bp, Glycine max
ttctgcacttgcgtccc

SEQ ID NO: 6 - DNA, 17 bp, Glycine max
tcaaaagcacttcgcca

SEQ ID NO: 7 - DNA, 17 bp, Glycine max
tactggaagttcgtgag

SEQ ID NO: 8 - DNA, 17 bp, Glycine max
cgaccttctcaacgcca

SEQ ID NO: 9 - DNA, 17 bp, Glycine max
tggaccaacagcaccat

SEQ ID NO: 10 - DNA, 17 bp, Glycine max
cggcgaggctgaggaga

SEQ ID NO: 11 - DNA, 17 bp, Glycine max
acttcgccacctcccaa

SEQ ID NO: 12 - DNA, 17 bp, Glycine max
tgcacttgcgtcccaca

SEQ ID NO: 13 - DNA, 17 bp, Glycine max
tgcacttggtttgtttg

SEQ ID NO: 14 - DNA, 17 bp, Glycine max
ttatatataagatagaa

SEQ ID NO: 15 - DNA, 17 bp, Glycine max
tatattataccatatca

SEQ ID NO: 16 - DNA, 17 bp, Glycine max
taactattatattatac

SEQ ID NO: 17 - DNA, 17 bp, Glycine max
atgtgtttctggggtta

SEQ ID NO: 18 - DNA, 659 bp, Glycine max
ataaatagccagcacacgggtctttggaaacgaagttgaaggggaaagagagaaaagaactagctagctggagtctgagaactctagcatatactagtactactacattccttcatcttcctttttatttttcaccataaaaaaacaagagcacaaagatggtgagagctccttgttgtgaaaagatgggtttgaagaagggtccttgggctaccgaggaagatcaaattctcacatcttacatccaaaaacatggccatggcaattggcgtgccctcccaaagcaagcaggtatttctatagttctataattacttcaatttaacaaatattataatatccaatttattatgacatgtttatacaccatagtatatgtataaatggcctagccttgtttagattttgacataatttagtactataaattaaacttgatggtccacaaagcttattgaatgtcatgatatgtgtattgattttttttctaatttctgtgataaattgctcaggcttgttaaggtgtgggaaaagctgtagactccgctggattaactatttgaggcctgatattaaaagagggaatttcacaattgaagaagaagaaactatcattaagctgcatgacatgctggggaacaggtatattcactgatcat

SEQ ID NO: 19 - DNA, 17 bp, Glycine max
taattaaaatattataa

SEQ ID NO: 20 - DNA, 17 bp, Glycine max
tttattggtactataaa

SEQ ID NO: 21 - DNA, 17 bp, Glycine max
taaaataaaatatacta

SEQ ID NO: 22 - DNA, 17 bp, Glycine max
atcttcatatttgataa

SEQ ID NO: 23 - DNA, 17 bp, Glycine max
taaaattaaattcttaa

SEQ ID NO: 24 - DNA, 17 bp, Glycine max
ataaaattaggtgtcaa

SEQ ID NO: 25 - DNA, 17 bp, Glycine max
aaattaggtgtcaatca

SEQ ID NO: 26 - DNA, 17 bp, Glycine max
tgctcaggcttgttaag

SEQ ID NO: 27 - DNA, 17 bp, Glycine max
agactccgctggattaa

SEQ ID NO: 28 - DNA, 17 bp, Glycine max
tccgctggattaactat

SEQ ID NO: 29 - DNA, 17 bp, Glycine max
taaaagagggaatttca

SEQ ID NO: 30 - DNA, 17 bp, Glycine max
aaagagggaatttcaca

SEQ ID NO: 31 - DNA, 17 bp, Glycine max
tggattaactatttgag

SEQ ID NO: 32 - DNA, 17 bp, Glycine max
gggaatttcacaattga

SEQ ID NO: 33 - DNA, 17 bp, Glycine max
ttcacaattgaagaaga

SEQ ID NO: 34 - DNA, 17 bp, Glycine max
tttcacaattgaagaag

SEQ ID NO: 35 - DNA, 17 bp, Glycine max
tgacatgctggggaaca

SEQ ID NO: 36 - DNA, 1541 bp, Glycine max
atgttgctggaacttgcacttggtttgtttgtgttagctttatctttgtttcaaaagcacttcgccacctcccaaaccctccaagcccaaagcctcgtcttcccttcattggccaccttcacctcttaaaagataaacttctccactatgcactcatcgatctctccaaaaagcatggccccttattctctctctccttcggctccatgccaaccgtcgttgcctccacccctgagttgttcaagctcttcctccaaacccacgaggcaacttccttcaacacaaggttccaaacctctgccataagacgcctcacttacgacaactctgtggccatggttccattcggaccttactggaagttcgtgaggaagctcatcatgaacgaccttctcaacgccaccaccgtcaacaagctcaggcctttgaggacccaacagatccgcaagttccttagggttatggcccaaagcgcagaggcccagaagccccttgacgtcaccgaggagcttctcaaatggaccaacagcaccatctccatgatgatgctcggcgaggctgaggagatcagagacatcgctcgcgaggttcttaagatcttcggcgaatacagcctcactgacttcatctggcctttgaagtatctcaaggttggaaagtatgagaagaggattgatgacatcttgaacaagttcgaccctgtcgttgaaagggtcatcaagaagcgccgtgagatcgtcagaaggagaaagaacggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaa

SEQ ID NO: 37 - DNA, 1532 bp, Glycine max
atgttgctggaacttgcacttggtttgtttgtgttagctttgtttctgcacttcgccacctcccaaaccctccaagcccaaagcctcgtcttcccttcattggccaccttcacctcttaaaagataaacttctccactatgcactcatcgatctctccaaaaagcatggccccttattctctctctccttcggctccatgccaaccgtcgttgcctccacccctgagttgttcaagctcttcctccaaacccacgaggcaacttccttcaacacaaggttccaaacctctgccataagacgcctcacttacgacaactctgtggccatggttccattcggaccttactggaagttcgtgaggaagctcatcatgaacgaccttctcaacgccaccaccgtcaacaagctcaggcctttgaggacccaacagatccgcaagttccttagggttatggcccaaagcgcagaggcccagaagccccttgacgtcaccgaggagcttctcaaatggaccaacagcaccatctccatgatgatgctcggcgaggctgaggagatcagagacatcgctcgcgaggttcttaagatcttcggcgaatacagcctcactgacttcatctggcctttgaagtatctcaaggttggaaagtatgagaagaggattgatgacatcttgaacaagttcgaccctgtcgttgaaagggtcatcaagaagcgccgtgagatcgtcagaaggagaaagaacggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaa

SEQ ID NO: 38 - DNA, 1562 bp, Glycine max
atgttgctggaacttgcacttggtttgtttgtgttagctttgtttctgcacttgcgtcccacagtgcaaaatcaaaagcacttcgccacctcccaaaccctccaagcccaaagcctcgtcttcccttcattggccaccttcacctcttaaaagataaacttctccactatgcactcatcgatctctccaaaaagcatggccccttattctctctctccttcggctccatgccaaccgtcgttgcctccacccctgagttgttcaagctcttcctccaaacccacgaggcaacttccttcaacacaaggttccaaacctctgccataagacgcctcacttacgacaactctgtggccatggttccattcggaccttactggaagttcgtgaggaagctcatcatgaacgaccttctcaacgccaccaccgtcaacaagctcaggcctttgaggacccaacagatccgcaagttccttagggttatggcccaaagcgcagaggcccagaagccccttgacgtcaccgaggagcttctcaaatggaccaacagcaccatctccatgatgatgctcggcgaggctgaggagatcagagacatcgctcgcgaggttcttaagatcttcggcgaatacagcctcactgacttcatctggcctttgaagtatctcaaggttggaaagtatgagaagaggattgatgacatcttgaacaagttcgaccctgtcgttgaaagggtcatcaagaagcgccgtgagatcgtcagaaggagaaagaacggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaa

SEQ ID NO: 39 - DNA, 1562 bp, Glycine max
atgttgcttgaacttgcacttggtttattggttttggctctgtttctgcacttgcgtcccacaccccaaaatcaaaagcacttcgccatctcccaaacccaccaagcccaaagcctcgtcttcccttcataggacaccttcatctcttaaaagacaaacttctccactacgcactcatcgacctctccaaaaaacatggtcccttattctctctctactttggctccatgccaaccgttgttgcctccacaccagaattgttcaagctcttcctccaaacgcacgaggcaacttccttcaacacaaggttccaaacctcagccataagacgcctcacctatgatagctcagtggccatggttcccttcggaccttactggaagttcgtgaggaagctcatcatgaacgaccttctcaacgccaccactgtaaacaagttgaggcctttgaggacccaacagatccgcaagttccttagggttatggcccaaggcgcagaggcacagaagccccttgacttgaccgaggagcttctgaaatggaccaacagcaccatctccatgatgatgctcggcgaggctgaggagatcagagacatcgctcgcgaggttcttaagatctttggcgaatacagcctcactgacttcatctggccattgaagcatctcaaggttggaaagtatgagaagaggatcgacgacatcttgaacaagttcgaccctgtcgttgaaagggtcatcaagaagcgccgtgagatcgtgaggaggagaaagaacggagaggttgttgagggtgaggtcagcggggttttccttgacactttgcttgaattcgctgaggatgagaccatggagatcaaaatcaccaaggaccacatcaagggtcttgttgtcgactttttctcggcaggaacagactccacagcggtggcaacagagtgggcattggcagaactcatcaacaatcctaaggtgttggaaaaggctcgtgaggaggtctacagtgttgtgggaaaggacagacttgtggacgaagttgacactcaaaaccttccttacattagagcaatcgtgaaggagacattccgcatgcacccgccactcccagtggtcaaaagaaagtgcacagaagagtgtgagattaatggatatgtgatcccagagggagcattgattctcttcaatgtatggcaagtaggaagagaccccaaatactgggacagaccatcggagttccgtcctgagaggttcctagagacaggggctgaaggggaagcagggcctcttgatcttaggggacaacattttcaacttctcccatttgggtctgggaggagaatgtgccctggagtcaatctggctacttcgggaatggcaacacttcttgcatctcttattcagtgcttcgacttgcaagtgctgggtccacaaggacagatattgaagggtggtgacgccaaagttagcatggaagagagagccggcctcactgttccaagggcacatagtcttgtctgtgttccacttgcaaggatcggcgttgcatctaaactcctttcttaa

SEQ ID NO: 40 - DNA, 1528 bp, Glycine max
atgttgcttgaacttgcacttggtttattggttttggctctgtttctgcacttgcgtcccaaacccaccaagcccaaagcctcgtcttcccttcataggacaccttcatctcttaaaagacaaacttctccactacgcactcatcgacctctccaaaaaacatggtcccttattctctctctactttggctccatgccaaccgttgttgcctccacaccagaattgttcaagctcttcctccaaacgcacgaggcaacttccttcaacacaaggttccaaacctcagccataagacgcctcacctatgatagctcagtggccatggttcccttcggaccttactggaagttcgtgaggaagctcatcatgaacgaccttctcaacgccaccactgtaaacaagttgaggcctttgaggacccaacagatccgcaagttccttagggttatggcccaaggcgcagaggcacagaagccccttgacttgaccgaggagcttctgaaatggaccaacagcaccatctccatgatgatgctcggcgaggctgaggagatcagagacatcgctcgcgaggttcttaagatctttggcgaatacagcctcactgacttcatctggccattgaagcatctcaaggttggaaagtatgagaagaggatcgacgacatcttgaacaagttcgaccctgtcgttgaaagggtcatcaagaagcgccgtgagatcgtgaggaggagaaagaacggagaggttgttgagggtgaggtcagcggggttttccttgacactttgcttgaattcgctgaggatgagaccatggagatcaaaatcaccaaggaccacatcaagggtcttgttgtcgactttttctcggcaggaacagactccacagcggtggcaacagagtgggcattggcagaactcatcaacaatcctaaggtgttggaaaaggctcgtgaggaggtctacagtgttgtgggaaaggacagacttgtggacgaagttgacactcaaaaccttccttacattagagcaatcgtgaaggagacattccgcatgcacccgccactcccagtggtcaaaagaaagtgcacagaagagtgtgagattaatggatatgtgatcccagagggagcattgattctcttcaatgtatggcaagtaggaagagaccccaaatactgggacagaccatcggagttccgtcctgagaggttcctagagacaggggctgaaggggaagcagggcctcttgatcttaggggacaacattttcaacttctcccatttgggtctgggaggagaatgtgccctggagtcaatctggctacttcgggaatggcaacacttcttgcatctcttattcagtgcttcgacttgcaagtgctgggtccacaaggacagatattgaagggtggtgacgccaaagttagcatggaagagagagccggcctcactgttccaagggcacatagtcttgtctgtgttccacttgcaaggatcggcgttgcatctaaactcctttcttaa

SEQ ID NO: 41 - DNA, 1562 bp, Glycine max
atgttgcttgaacttgcacttggtttattggttttggctctgtttctgcacttgcgtcccacaccgcaaaatcaaaagcacttcgccatctcccaaacccaccaagcccaaagcctcgtcttcccttcataggacaccttcatctcttaaaagacaaacttctccactacgcactcatcgacctctccaaaaaacatggtcccttattctctctctactttggctccatgccaaccgttgttgcctccacaccagaattgttcaagctcttcctccaaacgcacgaggcaacttccttcaacacaaggttccaaacctcagccataagacgcctcacctatgatagctcagtggccatggttcccttcggaccttactggaagttcgtgaggaagctcatcatgaacgaccttctcaacgccaccactgtaaacaagttgaggcctttgaggacccaacagatccgcaagttccttagggttatggcccaaggcgcagaggcacagaagccccttgacttgaccgaggagcttctgaaatggaccaacagcaccatctccatgatgatgctcggcgaggctgaggagatcagagacatcgctcgcgaggttcttaagatctttggcgaatacagcctcactgacttcatctggccattgaagcatctcaaggttggaaagtatgagaagaggatcgacgacatcttgaacaagttcgaccctgtcgttgaaagggtcatcaagaagcgccgtgagatcgtgaggaggagaaagaacggagaggttgttgagggtgaggtcagcggggttttccttgacactttgcttgaattcgctgaggatgagaccatggagatcaaaatcaccaaggaccacatcaagggtcttgttgtcgactttttctcggcaggaacagactccacagcggtggcaacagagtgggcattggcagaactcatcaacaatcctaaggtgttggaaaaggctcgtgaggaggtctacagtgttgtgggaaaggacagacttgtggacgaagttgacactcaaaaccttccttacattagagcaatcgtgaaggagacattccgcatgcacccgccactcccagtggtcaaaagaaagtgcacagaagagtgtgagattaatggatatgtgatcccagagggagcattgattctcttcaatgtatggcaagtaggaagagaccccaaatactgggacagaccatcggagttccgtcctgagaggttcctagagacaggggctgaaggggaagcagggcctcttgatcttaggggacaacattttcaacttctcccatttgggtctgggaggagaatgtgccctggagtcaatctggctacttcgggaatggcaacacttcttgcatctcttattcagtgcttcgacttgcaagtgctgggtccacaaggacagatattgaagggtggtgacgccaaagttagcatggaagagagagccggcctcactgttccaagggcacatagtcttgtctgtgttccacttgcaaggatcggcgttgcatctaaactcctttcttaa

SEQ ID NO: 42 - DNA, 1561 bp, Glycine max
atgttgcttgaacttgcacttggtttattggttttggctctgtttctgcacttgcgtcccacacccaaaatcaaaagcacttcgccatctcccaaacccaccaagcccaaagcctcgtcttcccttcataggacaccttcatctcttaaaagacaaacttctccactacgcactcatcgacctctccaaaaaacatggtcccttattctctctctactttggctccatgccaaccgttgttgcctccacaccagaattgttcaagctcttcctccaaacgcacgaggcaacttccttcaacacaaggttccaaacctcagccataagacgcctcacctatgatagctcagtggccatggttcccttcggaccttactggaagttcgtgaggaagctcatcatgaacgaccttctcaacgccaccactgtaaacaagttgaggcctttgaggacccaacagatccgcaagttccttagggttatggcccaaggcgcagaggcacagaagccccttgacttgaccgaggagcttctgaaatggaccaacagcaccatctccatgatgatgctcggcgaggctgaggagatcagagacatcgctcgcgaggttcttaagatctttggcgaatacagcctcactgacttcatctggccattgaagcatctcaaggttggaaagtatgagaagaggatcgacgacatcttgaacaagttcgaccctgtcgttgaaagggtcatcaagaagcgccgtgagatcgtgaggaggagaaagaacggagaggttgttgagggtgaggtcagcggggttttccttgacactttgcttgaattcgctgaggatgagaccatggagatcaaaatcaccaaggaccacatcaagggtcttgttgtcgactttttctcggcaggaacagactccacagcggtggcaacagagtgggcattggcagaactcatcaacaatcctaaggtgttggaaaaggctcgtgaggaggtctacagtgttgtgggaaaggacagacttgtggacgaagttgacactcaaaaccttccttacattagagcaatcgtgaaggagacattccgcatgcacccgccactcccagtggtcaaaagaaagtgcacagaagagtgtgagattaatggatatgtgatcccagagggagcattgattctcttcaatgtatggcaagtaggaagagaccccaaatactgggacagaccatcggagttccgtcctgagaggttcctagagacaggggctgaaggggaagcagggcctcttgatcttaggggacaacattttcaacttctcccatttgggtctgggaggagaatgtgccctggagtcaatctggctacttcgggaatggcaacacttcttgcatctcttattcagtgcttcgacttgcaagtgctgggtccacaaggacagatattgaagggtggtgacgccaaagttagcatggaagagagagccggcctcactgttccaagggcacatagtcttgtctgtgttccacttgcaaggatcggcgttgcatctaaactcctttcttaa

SEQ ID NO: 43 - DNA, 1561 bp, Glycine max
atgttgcttgaacttgcacttggtttattggttttggctctgtttctgcacttgcgtcccacacgcaaaatcaaaagcacttcgccatctcccaaacccaccaagcccaaagcctcgtcttcccttcataggacaccttcatctcttaaaagacaaacttctccactacgcactcatcgacctctccaaaaaacatggtcccttattctctctctactttggctccatgccaaccgttgttgcctccacaccagaattgttcaagctcttcctccaaacgcacgaggcaacttccttcaacacaaggttccaaacctcagccataagacgcctcacctatgatagctcagtggccatggttcccttcggaccttactggaagttcgtgaggaagctcatcatgaacgaccttctcaacgccaccactgtaaacaagttgaggcctttgaggacccaacagatccgcaagttccttagggttatggcccaaggcgcagaggcacagaagccccttgacttgaccgaggagcttctgaaatggaccaacagcaccatctccatgatgatgctcggcgaggctgaggagatcagagacatcgctcgcgaggttcttaagatctttggcgaatacagcctcactgacttcatctggccattgaagcatctcaaggttggaaagtatgagaagaggatcgacgacatcttgaacaagttcgaccctgtcgttgaaagggtcatcaagaagcgccgtgagatcgtgaggaggagaaagaacggagaggttgttgagggtgaggtcagcggggttttccttgacactttgcttgaattcgctgaggatgagaccatggagatcaaaatcaccaaggaccacatcaagggtcttgttgtcgactttttctcggcaggaacagactccacagcggtggcaacagagtgggcattggcagaactcatcaacaatcctaaggtgttggaaaaggctcgtgaggaggtctacagtgttgtgggaaaggacagacttgtggacgaagttgacactcaaaaccttccttacattagagcaatcgtgaaggagacattccgcatgcacccgccactcccagtggtcaaaagaaagtgcacagaagagtgtgagattaatggatatgtgatcccagagggagcattgattctcttcaatgtatggcaagtaggaagagaccccaaatactgggacagaccatcggagttccgtcctgagaggttcctagagacaggggctgaaggggaagcagggcctcttgatcttaggggacaacattttcaacttctcccatttgggtctgggaggagaatgtgccctggagtcaatctggctacttcgggaatggcaacacttcttgcatctcttattcagtgcttcgacttgcaagtgctgggtccacaaggacagatattgaagggtggtgacgccaaagttagcatggaagagagagccggcctcactgttccaagggcacatagtcttgtctgtgttccacttgcaaggatcggcgttgcatctaaactcctttcttaa

SEQ ID NO: 44 - DNA, 186 bp, Glycine max
  variation (17)..(27): /replace=“ ”
  misc_feature (17)..(27): /note=“This region may or may not be present in its entirety”
  variation (32)..(32): /replace=“ ”
  variation (38)..(39): /replace=“ ”
  misc_feature (38)..(39): /note=“This region may or may not be present in its entirety”
  SITE (1)..(186): /note=“Variant nucleotides given in the sequence have no preference with respect to those in the annotations for variant positions”
rgatcamasaaaccaacaacagttcttgsactgagrwttcacgatgttgctkgaacttgcacttggtttrttkgtkttrgctytgtttctgcacttgcgtcccacaccmastgcaaaatcaaaagcacttcgccayctcccaaacccwccaagcccaaagcctcgtcttcccttcatwggmcacct

SEQ ID NO: 45 - DNA, 186 bp, Glycine max
ggatcacagaaaccaacaacagttcttgcactgaggtttcacgatgttgctggaacttgcacttggtttgtttgtgttagctttgtttctgcacttgcgtcccacaccaagtgcaaaatcaaaagcacttcgccacctcccaaaccctccaagcccaaagcctcgtcttcccttcattggccacct

SEQ ID NO: 46 - DNA, 172 bp, Glycine max
agatcaaacaaaccaaggacgagaacacgatgttgcttgaacttgcacttggtttattggttttggctctgtttctgcacttgcgtcccacacccactgcaaaatcaaaagcacttcgccatctcccaaacccaccaagcccaaagcctcgtcttcccttcataggacacct

SEQ ID NO: 47 - DNA, 71 bp, Glycine max
rgctytgtttctgcacttgcgtcccacaccmastgcaaaatcaaaagcacttcgccayctcccaaacccwc

SEQ ID NO: 48 - DNA, 71 bp, Glycine max
agctttgtttctgcacttgcgtcccacaccaagtgcaaaatcaaaagcacttcgccacctcccaaaccctc

SEQ ID NO: 49 - DNA, 71 bp, Glycine max
ggctctgtttctgcacttgcgtcccacacccactgcaaaatcaaaagcacttcgccatctcccaaacccac

SEQ ID NO: 50 - DNA, 721 bp, Glycine max
ggaatatccaaggataaatagccagcacacgggtctttggaaacgaagttgaaggggaaagagagaaaagaactagctagctggagtctgagaactctagcatgtactagtactactcctacattcctccatcttcctttgtactattttttttttaaccataaaagcaagagcacaaagatggtgagagctccttgttgtgaaaagatgggtttgaagaagggtccttgggctcccgaggaagatcaaatcctcacatcttacattgacaaacatggtcatggcaattggcgtgccctcccaaagcaagcaggtacatctatagttctattacttcaatttaacaaatattataatatccaatttattatgacatgtttatacaccatagtatatgtataaatggcctagccttgtttagattttgacataatttagtactataaattaaacttgatggtccacaaagcttattgaatgtcatgatatgtgtattgattttttttctaatttctgtgataaattgctcaggcttgttaaggtgtgggaaaagctgtagactccgctggattaactatttgaggcctgatattaaaagagggaatttcacaattgaagaagaagaaactatcattaagctgcatgacatgctggggaacaggtatattcactgatcatttccttgctttggacccatatgacattaatcatgctaattaac

SEQ ID NO: 51 - DNA, 80 bp, Glycine max
attttttacaagataaaattaaattcttaactacctgttaagagataaaattaggtgtcaatcatattatgacatgctag

(19) United States
(12) Patent Application Publication
(10) Pub. No.: US 2022/0380787 A1
(43) Pub. Date: Dec. 1, 2022

(54) LOW PHYTOESTROGEN SOYBEAN BASED ON GENE EDITING

(71) Applicant: Calyxt, Inc., Roseville, MN (US)

(72) Inventors: Jim PRESNAIL, Minneapolis, MN (US); Zachary DEMOREST, Roseville, MN (US); Qiwei SHAN, Roseville, MN (US)

(21) Appl. No.: 17/753,918

(22) PCT Filed: Sep. 18, 2020
(86) PCT No.: PCT/US2020/051616

§ 371 Date: Mar. 18, 2022

Related U.S. Application Data
(60) Provisional application No. 62/903,484, filed on Sep. 20, 2019.

(51) Int. Cl. C12N 15/82

(52) U.S. Cl. CPC C12N 15/8243; C12N 15/8213

(57) ABSTRACT

Gene-edited soybean plants, plant parts, and plant cells that have a reduced isoflavone content compared to the corresponding unaltered soybean plant, plant part, or plant cell and methods of their production are disclosed. For example, introducing one or more mutations in the coding region of Myb29 gene, the MYBCORE binding motif of the IFS2 promoter, the MYBCORE binding motif of the IFS1 promoter, the coding region of the IFS2 gene, the coding region of the IFS1 gene, or a combination thereof is used to produce the gene-edited plant, plant part or plant cell via targeted genome modification.

### SEQUENCE LISTING

The instant application contains a Sequence Listing which has been submitted electronically in ASCII format and is hereby incorporated by reference in its entirety. Said ASCII copy, created on Sep. 18, 2020, is named 1702_042PCT1_SL.txt and is 33,318 bytes in size.

### BACKGROUND

Soybean is an economical and abundant source of protein for human consumption. Protein extracts derived from soybean meal have a favorable amino acid profile and high protein content. Soybean extract, as well as whole soybean, are used in a wide variety of consumer products. Soybean-extracted protein is an important component of infant formula; formulations containing soy protein are valuable for example, for infants with lactose intolerance.

Plants produce a wide variety of secondary chemicals, along with the fundamental molecules critical to normal plant development and physiology. Isoflavones are a category of secondary plant chemical compounds produced by legumes with a wide variety of functions. Isoflavones are also described as phytoestrogens because they are plant-derived estrogen like compounds. As such, isoflavones have been investigated for their potential impacts on human hormonal and developmental systems. Soybean protein extracts also contain secondary plant metabolites including a variety of isoflavones (specifically genistein and daidzein), which are beneficial to consumer populations such as mature women, with potential benefits in prevention of menopausal symptoms. These estrogen-like isoflavones, however, are potentially less desirable for other populations, including infants and consumers of protein supplements.

Production of isoflavonoids represents a branch from the phenylpropanoid pathway of legumes which also contains the biosynthetic pathways for several other important plant chemicals including lignins, anthocyanins, and stilbenes. Isoflavones are known to be involved in interactions with other organisms including nodulation of Rhizobacteria and pathogen response.

Alterations in isoflavone levels in plants have been approached using techniques such as targeted induced local lesions in genomes (TILLING), transgenic overexpression, or transgenic suppression (see, e.g., U.S. Pat. Nos. 8,329,995, 5,994,075, 7,098,011). However, these methods fail to deliver a product free of unintended mutations or transgenic DNA sequences and fail to meet consumer demand and public acceptance.

A need exists for soybean plants that have a lower content of isoflavones compared to the existing varieties.

### SUMMARY

The aspects of the invention involve targeted mutagenesis methods, specifically genome editing, and exclude embodiments that are solely based on generating plants by traditional breeding methods (i.e., the plants and plant material products (including seed) are not exclusively obtained by means of an essentially biological process and the process features do not define an essentially biological process). As explained herein, the lower or reduced content of isoflavones in the soybean plants, plant parts, and plant cells disclosed herein is not due to the presence of a transgene.

In one aspect, provided herein is a soybean plant, plant part, or plant cell having reduced isoflavone content compared to the unaltered soybean plant, plant part, or plant cell, wherein the soybean plant, plant part, or plant cell comprises one or more mutations in the coding region of one or both IFS1 and IFS2 genes, and wherein the plant, plant part, or plant cell is characterized by reduced expression of the one or both IFS1 and IFS2 genes.

In another aspect, provided herein is a soybean plant, plant part, or plant cell having reduced isoflavone content compared to an unaltered soybean plant, plant part, or plant cell, wherein the soybean plant, plant part, or plant cell comprises one or more mutations in the coding region of mutation in the coding region of Myb29 gene, and wherein the plant, plant part, or plant cell is characterized by reduced expression reduced expression of one or both IFS2 gene and IFS1 genes.

In another aspect, provided herein is a soybean plant, plant part, or plant cell having reduced isoflavone content compared to an unaltered soybean plant, plant part, or plant cell, wherein the soybean plant, plant part, or plant cell comprises one or more mutations in the coding region of MYBCORE binding motif of the IFS1 promoter or the IFS2 promoter and wherein the plant, plant part, or plant cell is characterized by reduced expression reduced expression of one or both IFS2 gene and IFS1 genes.

In some embodiments of the soybean plant, plant part, or plant cell disclosed herein, the one or more mutations is introduced using a targeted genome modification using a rare-cutting endonuclease. In some embodiments, the rare-cutting endonuclease is a transcription activator-like effector nuclease (TALEN). In some embodiments, the one or more mutations is introduced using a TALEN comprising a TAL effector which binds to a sequence within SEQ ID NO: 1 or SEQ ID NO: 2. In some embodiments, the one or more mutations is introduced using one or more TALENs comprising a TAL effector which binds to a sequence within SEQ ID NO: 3 or SEQ ID NO: 4. In some embodiments, the one or more mutations is introduced using one or more TALENs comprising a TAL effector which binds to a sequence within SEQ ID NO: 18. In some embodiments, the one or more mutations is introduced using one or more TALENs comprising a TAL effector which binds to one or more of SEQ ID NO: 5-17 and 19-35.

In some embodiments of the soybean plant, plant part, or plant cell described herein, the plant, plant part, or plant cell does not comprise a transgene. In some embodiments, the plant part is seed.

In some embodiments, the plant, plant part, or plant cell has a content of isoflavones that is reduced by about 10%, about 20%, about 30%, about 50%, about 60%, about 70%, about 80%, or about 90% compared to the content of isoflavones in the corresponding unaltered soybean plant, plant part, or plant cell.

In another aspect, provided herein is a method for producing a soybean plant, plant part, or plant cell with decreased content of isoflavones compared to an unaltered plant, plant part, or plant cell using targeted genome modification comprising introducing one or more mutations into the coding region of in the coding region of Myb29 gene, the MYBCORE binding motif of the IFS2 promoter, the MYBCORE binding motif of the IFS1 promoter, the coding region of the IFS2 gene, the coding region of the IFS1 gene, or a combination thereof.

In some embodiments, the mutation is introduced using a rare-cutting endonuclease. In some embodiments, the rare-cutting endonuclease is a TALEN. In some embodiments, the one or more mutations is introduced using a TALEN comprising a TAL effector which binds to a sequence within SEQ ID NO: 1 or SEQ ID NO: 2. In some embodiments, the one or more mutations is introduced using a TALEN comprising a TAL effector which binds to a sequence within SEQ ID NO: 3 or SEQ ID NO: 4. In some embodiments, the one or more mutations is introduced using a TALEN comprising a TAL effector which binds to a sequence within SEQ ID NO: 18. In some embodiments, the one or more mutations is introduced using a TALEN comprising a TAL effector which binds to one or more of SEQ ID NO: 5-17 and 19-35.

In some embodiments of the methods disclosed herein, the soybean plant, plant part, or plant cell does not comprise a transgene.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-B shows coding sequences of: (A) soybean IFS1 (SEQ ID NO: 1) and (B) soybean IFS2 (SEQ ID NO: 2).

FIG. 2 shows IFS sequence targeted by left and right half TALENs flanking the target sequence (greater resolution shown in exploded view). Figure discloses SEQ ID NOS 44-49, respectively, in order of appearance.

FIG. 3 is an illustration of Myb29 transcription factor sequence and associated TALENs. Figure discloses SEQ ID NO: 50.

FIG. 4 shows IFS1 promoter sequence (SEQ. ID NO: 3) with four unique MYBCORE motifs (CNGTTR) highlighted.

FIG. 5 is an illustration of IFS1 promoter with MYBCORE motif.

FIG. 6 shows IFS2 promoter sequence (SEQ. ID NO: 4) with identified MYBCORE motif.

FIG. 7 is an illustration of IFS2 promoter with MYBCORE and TALEN binding sites. Figure discloses SEQ ID NO: 51.

FIG. 8 shows a coding sequence of Myb29 (SEQ. ID NO: 18).

FIG. 9 shows a representative IFS1 DNA sequence of a confirmed mutant profile (−25) from a regenerated T₀ plant. Figure discloses SEQ ID NO: 36.

FIG. 10 shows a representative IFS1 DNA sequence of a confirmed mutant profile (−34) from a regenerated T₀ plant. Figure discloses SEQ ID NO: 37.

FIG. 11 shows a representative IFS1 DNA sequence of a confirmed mutant profile (−4) from a regenerated T₀ plant. Figure discloses SEQ ID NO: 38.

FIG. 12 shows a representative IFS2 DNA sequence of a confirmed mutant profile (−4) from a regenerated T₀ plant. Figure discloses SEQ ID NO: 39.

FIG. 13 shows a representative IFS2 DNA sequence of a confirmed mutant profile (−38) from a regenerated T₀ plant. Figure discloses SEQ ID NO: 40.

FIG. 14 shows a representative IFS2 DNA sequence of a confirmed mutant profile (−4), designated “IFS2.2”, from a regenerated T₀ plant. The “IFS2.2” designation indicates a unique variant. In this case, the deletion site is distinct from the −4 nt deletion mutant described in FIG. 12. Figure discloses SEQ ID NO: 41.

FIG. 15 shows a representative IFS2 DNA sequence of a confirmed mutant profile (−5) from a regenerated T₀ plant. Figure discloses SEQ ID NO: 42.

FIG. 16 shows a representative IFS2 DNA sequence of a confirmed mutant profile (−5), designated “IFS2.2”, from a regenerated T₀ plant. The deletion site is distinct from the −5 nt deletion mutant described in FIG. 15. Figure discloses SEQ ID NO: 43.

FIG. 17 shows the results of phytoestrogen analysis comparing the amounts of daidzin, glycitin, and genistin extracted from the seeds of three mutant plant lines (designated #1-#3) to the wild type seed extract (Control) expressed as μg/mg of dry plant material.

FIG. 18 is a graphical representation of the values provided in FIG. 17.

FIG. 19 shows the results of phytoestrogen content analysis comparing the total phytoestrogen amount extracted from the seeds of three mutant plant lines (designated #1-#3) to the wild type seed extract (Control) expressed as μg/mg of dry plant material.

FIG. 20 shows representative HPLC chromatograms of isoflavone components in control extract (upper) and Sample #1 extract (lower). The boxed area shows the absorbance peaks associated with daidzin, glycitin, and genistin, respectively, are significantly larger in the control compared with Sample #1.

### DETAILED DESCRIPTION

The present disclosure focuses on the selective targeting of the ultimate biosynthetic enzymes responsible for production of isoflavones, the isoflavone synthases (IFS1 and IFS2) as well as genes (such as Myb29) and regulatory sequences (such as promoters) that regulate expression of IFS1 and IFS2.

The present disclosure addresses consumer needs for soybean protein and oil products with lower isoflavone content. The present disclosure further addresses a consumer need for non-transgenic soybean protein and oil products with lower isoflavone content.

Thus, in one aspect, provided herein are soybean plants, plant parts, and plant cells having reduced isoflavone content compared to an unaltered soybean plant, plant part, or plant cell, the soybean plants, plant parts, and plant cells of the present disclosure comprising one or more mutations in the coding regions of isoflavone synthase (IFS) genes, IFS1 and IFS2. The soybean plants, plant parts, and plant cells disclosed herein are transgene-free and free of modifications in genes with homology to the targets IFS1 and IFS2.

In another aspect, provided herein are soybean plants, plant parts, and plant cells having reduced isoflavone content compared to an unaltered soybean plant, plant part, or plant cell, the soybean plants, plant parts, and plant cells of the present disclosure comprising one or more mutations in the gene Myb29, a transcription regulatory factor important in the controlled expression of IFS1 and IFS2.

In another aspect, provided herein are soybean plants, plant parts, and plant cells having reduced isoflavone content compared to the unaltered soybean plant, plant part, or plant cell comprising one or more mutations in the regulatory region of the IFS1 and/or IFS2. In some embodiments, the regulatory region is a promoter region.

In another aspect, provided herein is a method of producing a soybean plant, plant part, or plant cell having reduced isoflavone content compared to an unaltered soybean plant, plant part, or plant cell, the method comprising targeted gene editing by introducing one or more mutations into the coding region of the IFS1, the coding region of IFS2, the regulatory region of the IFS1 gene, the regulatory region of the IFS2 gene, the gene Myb29, or a combination thereof, wherein the expression of IFS1 and/or IFS2 genes is eliminated. In some embodiments, the gene editing is performed using a TALEN editing of the genes in order to eliminate the expression of IFS1 and/or IFS2 genes in the soybean seed without disrupting expression in the soybean roots where isoflavones are beneficial to the soybean.

The inventors discovered that IFS1 and IFS2 gene sequences have regions that can be simultaneously targeted by a single rare-cutting endonuclease such as TALEN. The inventors further analyzed isoflavone synthase gene sequences from several soybean varieties and identified conserved regions to further focus the target sites of the rare-cutting endonucleases, such as TALENs. Thus, the rare-cutting endonucleases, such as TALENs, validated for the gene editing of IFS1 and IFS2 can be used in a wide variety of commercial soybean lines. Plants generated downstream of TALEN editing have demonstrably reduced isoflavones than standard soybean varieties grown under similar conditions. In some embodiments, the soybean plant, plant part, or plant cell disclosed herein has a content of isoflavones that is reduced by about 10%, about 20%, about 30%, about 50%, about 60%, about 70%, about 80%, about 90%, about 95%, about 98% or about 99% compared to the content of isoflavones in the corresponding unaltered soybean plant, plant part, or plant cell. As used herein, an “unaltered soybean” refers to a soybean that has not been altered by gene editing techniques, e.g. TALEN editing disclosed herein. Unaltered plants include wild-type plants. In some embodiments, the soybean plant part is soybean seed.

Thus, the present disclosure provides a previously unknown method for specifically targeting the isoflavone synthase genes across multiple varieties of soybean in a manner that does not result in mutations in other locations throughout the genome, and without introduction of foreign material in the final selected edited lines. Furthermore, plants derived from gene-editing technologies can be advantageously crossed to elite lines that are recalcitrant to cell biology regeneration.

Expression of the IFS1 and IFS2 genes has been shown to be regulated at least in part by the Myb29 transcription factor (Chu S. et al., PLoS Genet. 13(5) (2017)). Myb29 is a transcriptional activator that binds to MYBCORE sequence motifs in promoter regions and upregulates expression of the downstream gene, such as IFS1/IFS2. Thus, disclosed herein are methods of producing a soybean plant, plant part, or plant cell by introduction of mutations in the Myb29 gene, thereby reducing or eliminating expression of one or more of the IFS1 and IFS2 genes. Additionally, disclosed herein is a soybean plant, plant part, or plant cell comprising one or more mutations in the Myb29 gene wherein the soybean plant, plant part, or plant cell has a reduced or eliminated expression of one or more of the IFS1 and IFS2 genes.

The inventors identified MYBCORE sequences in the promoter regions of IFS1 and IFS2 and TALENs designed to ablate these binding motifs. Ablation of MYBCORE binding motifs from IFS1 and/or IFS2 promoters is of particular interest because the Myb29 gene can be left unaltered and fully functional, able to regulate other target genes while eliminating its regulation of IFS1 and/or IFS2.

In some embodiments of the methods, plants, plant parts, and plant cells disclosed herein, the one or more mutations is introduced via a targeted genome modification using a rare-cutting endonuclease. The term “rare-cutting endonucleases” herein refer to natural or engineered proteins having endonuclease activity directed to nucleic acid sequences having a recognition sequence (target sequence) about 12-40 bp in length (e.g., 14-40 bp in length). Typical rare-cutting endonucleases cause cleavage inside their recognition site, leaving 4 nucleotides staggered cut with 3′OH or 5′OH overhangs. These rare-cutting endonucleases may be meganucleases, such as wild type or variant proteins of homing endonucleases, more particularly belonging to the dodecapeptide family (see, WO 2004/067736) or may result from fusion proteins that associate a DNA-binding domain and a catalytic domain with cleavage activity. TAL-effector endonucleases and zinc-finger-nucleases (ZFN) are examples of fusions of DNA-binding domains with the catalytic domain of the endonuclease Fokl. For a review of rare-cutting endonucleases see Baker, Nature Methods 9:23-26, 2012.

“Rare-cutting endonucleases” also encompass RNA-guided systems that can be used in the methods provided herein. For example, the clustered regularly interspaced short palindromic repeats/CRISPR-associated (CRISPR/Cas) systems use RNA to direct DNA cleavage (see, e.g., Belahj et al., Plant Methods 9:39, 2013). These systems consist of a Cas9 endonuclease and a guide RNA (either a complex between a CRISPR RNA (crRNA) and trans-activating crRNA (tracrRNA), or a synthetic fusion between the 3′ end of the crRNA and 5′ end of the tracrRNA). The guide RNA directs Cas9 binding and DNA cleavage to sequences that are adjacent to a proto-spacer adjacent motif (PAM), e.g., NGG for Cas9 from Streptococcus pyogenes. Once at the target DNA sequence, Cas9 generates a DNA double-strand break at a position three nucleotides from the 3′ end of the crRNA sequence that is complementary to the target sequence. In addition to Cas9, the CRISPR system from Prevotella and Francisella 1 (Cpf1) can be employed to introduce mutations (see, for example, Zetsche et al., Cell 163:759-771 (2015)).

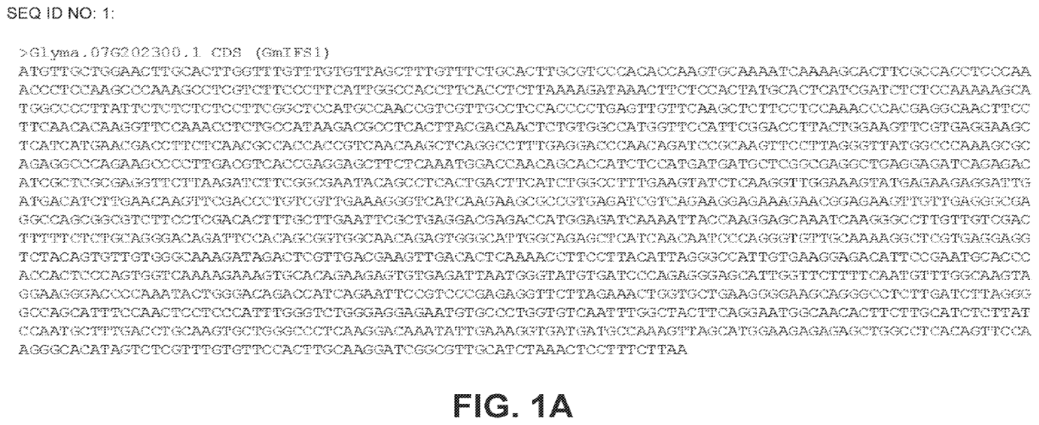

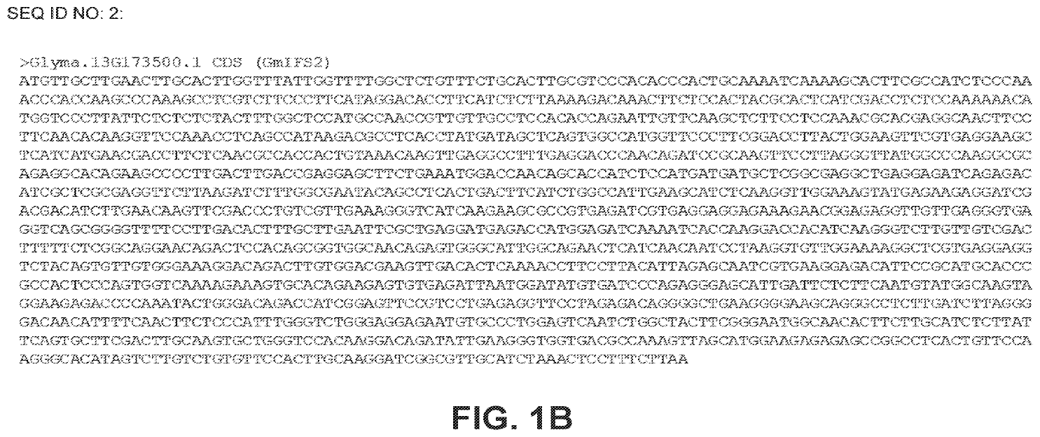

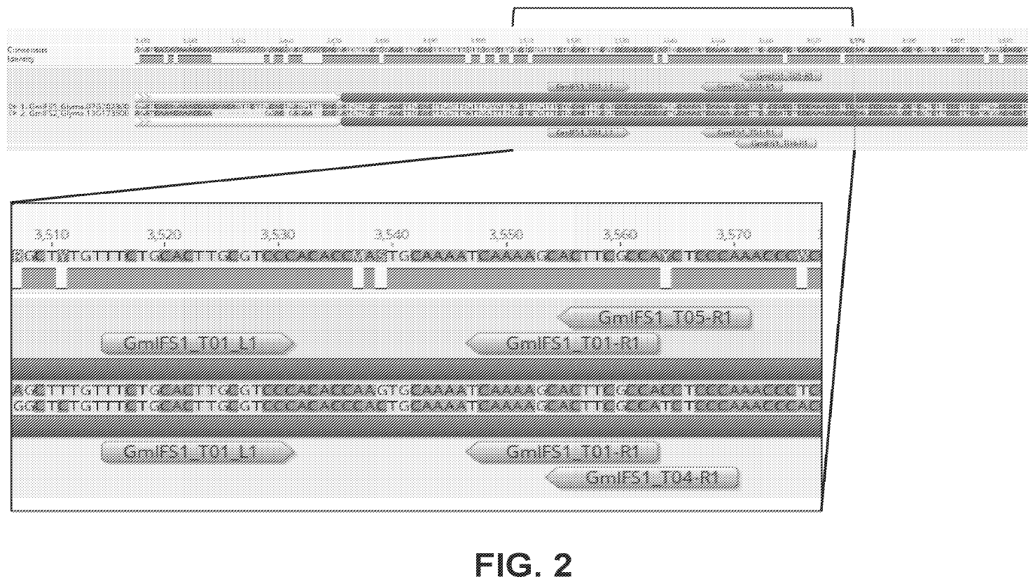

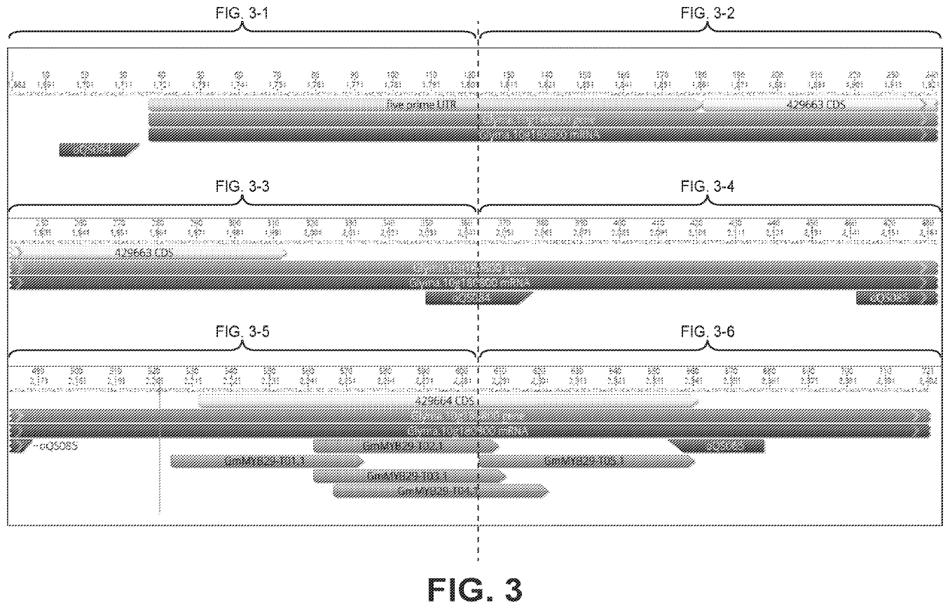

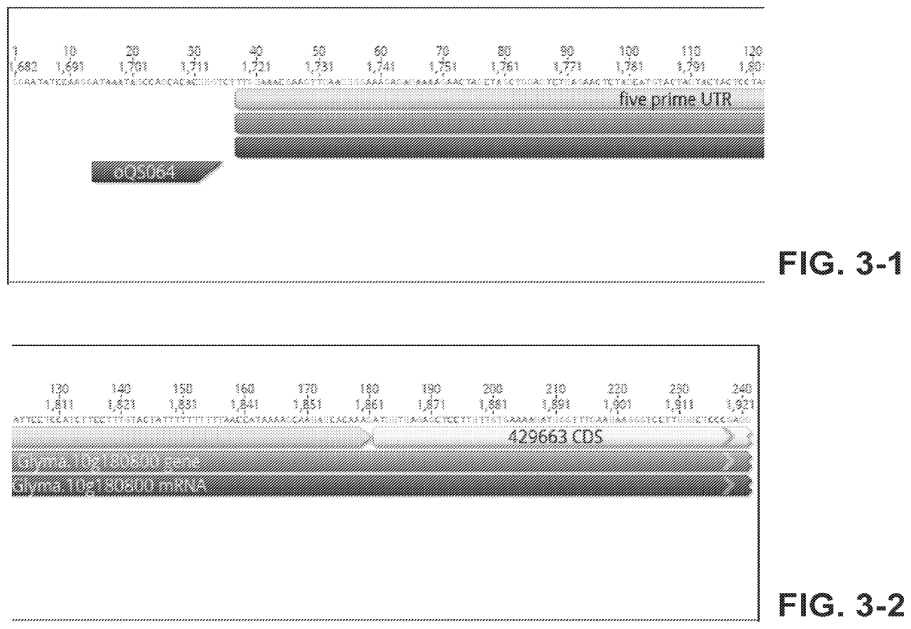

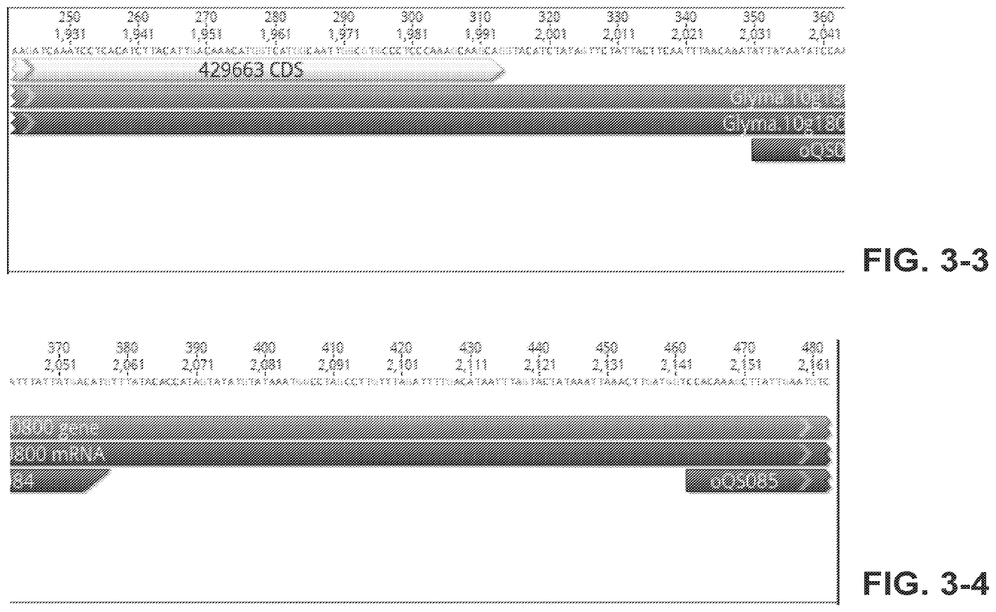

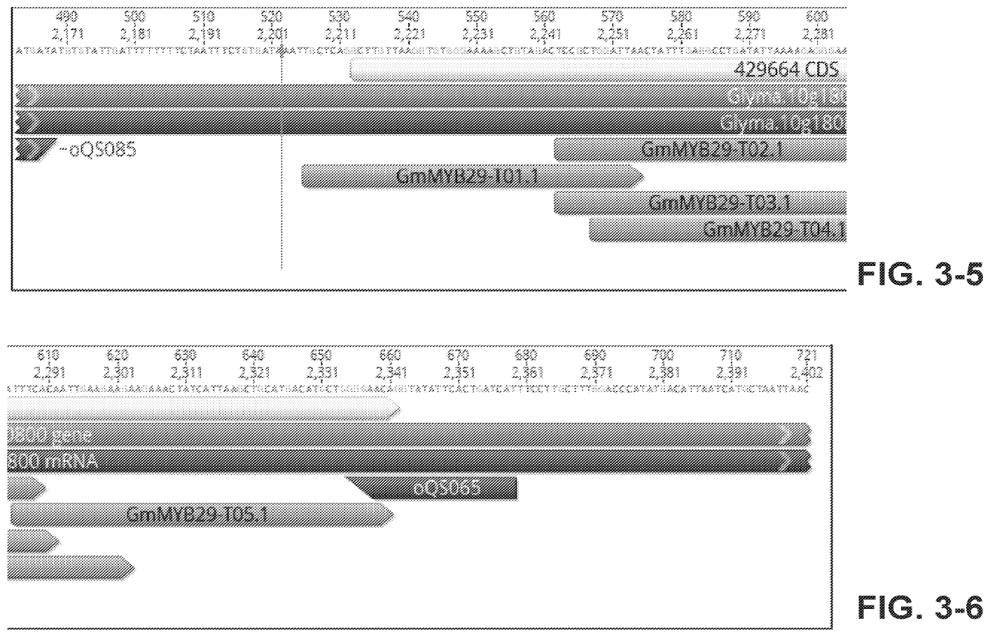

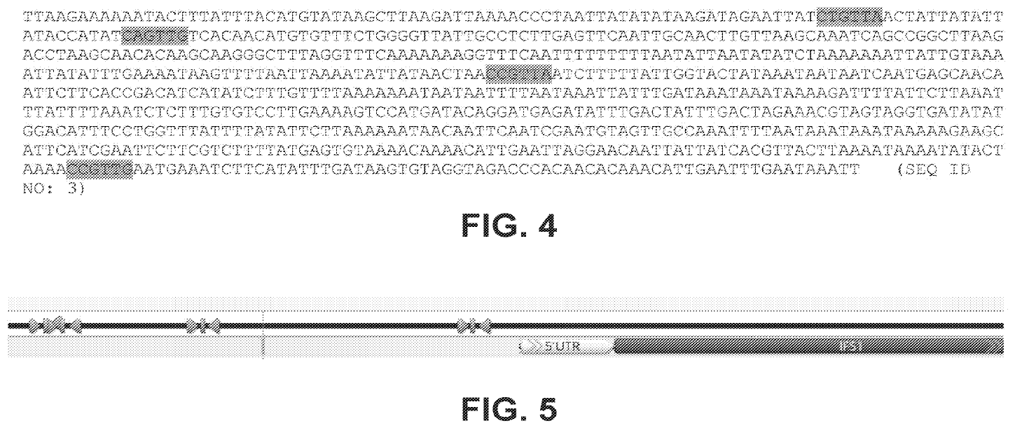

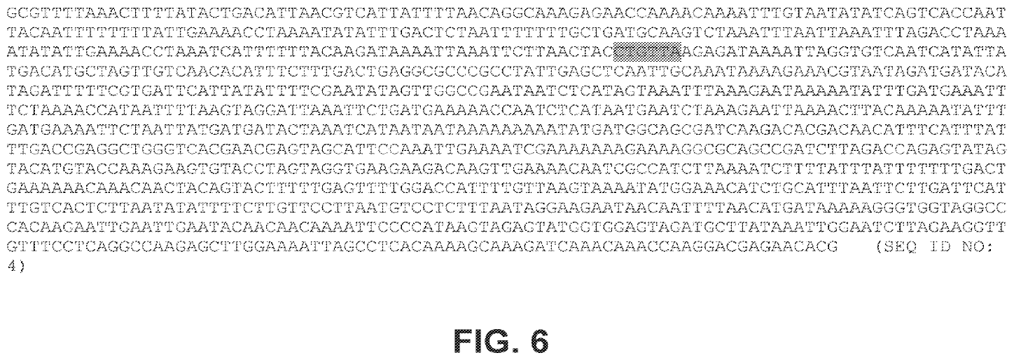

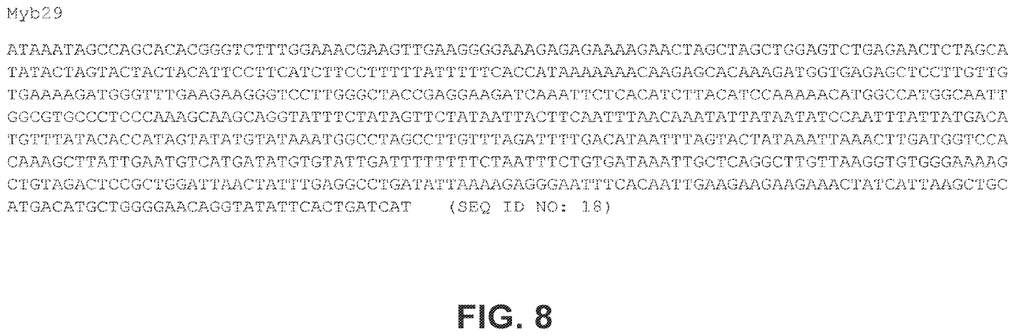

In some embodiments of the methods, plants, plant parts, and plant cells disclosed herein, the one or more mutations is introduced using a TALEN comprising a TAL effector which binds to a sequence within SEQ ID NO: 1 (FIG. 1A). In some embodiments of the methods, plants, plant parts, and plant cells disclosed herein, the one or more mutations is introduced using a TALEN comprising a TAL effector which binds to a sequence within SEQ ID NO: 2 (FIG. 1B). In some embodiments of the methods, plants, plant parts, and plant cells disclosed herein, the one or more mutations are introduced using a TALEN comprising a TAL effector which binds to a sequence within SEQ ID NO: 3 (FIG. 4). In some embodiments of the methods, plants, plant parts, and plant cells disclosed herein, the one or more mutations is introduced using a TALEN comprising a TAL effector which binds to a sequence within SEQ ID NO: 4 (FIG. 6). In some embodiments of the methods, plants, plant parts, and plant cells disclosed herein, the one or more mutations is introduced using a TALEN comprising a TAL effector which binds to a sequence within SEQ ID NO: 18 (FIG. 8) In some embodiments, the one or more mutations is introduced using one or more TALEN comprising a TAL effector which binds to a sequence selected from SEQ ID NO: 5-17 and 19-35 shown in Table 1 below.

TABLE 1
TAL effector endonuclease target sequences

| SEQ ID NO: | SEQ Name | Sequence |
|---|---|---|
| 5 | GmIFS1_T01_L1 | TTCTGCACTTGCGTCCC |
| 6 | GmIFS1_T01-R1 | TCAAAAGCACTTCGCCA |
| 7 | GmIFS1_T02-L1 | TACTGGAAGTTCGTGAG |
| 8 | GmIFS1_T02-R1 | CGACCTTCTCAACGCCA |
| 9 | GmIFS1_T03-L1 | TGGACCAACAGCACCAT |
| 10 | GmIFS1_T03-R1 | CGGCGAGGCTGAGGAGA |
| 11 | GmIFS1_T05-R1 | ACTTCGCCACCTCCCAA |
| 12 | GmIFS1_T06-R1 | TGCACTTGCGTCCCACA |
| 13 | GmIFS1_T07-L1 | TGCACTTGGTTTGTTTG |
| 14 | GmIFS1_MYB_T01-L1 | TTATATATAAGATAGAA |
| 15 | GmIFS1_MYB_T01-R1 | TATATTATACCATATCA |
| 16 | GmIFS1_MYB_T02-L1 | TAACTATTATATTATAC |
| 17 | GmIFS1_MYB_T02-R1 | ATGTGTTTCTGGGGTTA |
| 19 | GmIFS1_MYB_T03-L1 | TAATTAAAATATTATAA |
| 20 | GmIFS1_MYB_T03-R1 | TTTATTGGTACTATAAA |
| 21 | GmIFS1_MYB_T04-L1 | TAAAATAAAATATACTA |
| 22 | GmIFS1_MYB_T04-R1 | ATCTTCATATTTGATAA |
| 23 | GmIFS2_MYB_T01-L1 | TAAAATTAAATTCTTAA |
| 24 | GmIFS2_MYB_T01-R1 | ATAAAATTAGGTGTCAA |
| 25 | GmIFS2_MYB_T01-R2 | AAATTAGGTGTCAATCA |
| 26 | GmMYB29_T01-L1 | TGCTCAGGCTTGTTAAG |
| 27 | GmMYB29_T01-R1 | AGACTCCGCTGGATTAA |
| 28 | GmMYB29_T02-L1 | TCCGCTGGATTAACTAT |
| 29 | GmMYB29_T02-R1 | TAAAAGAGGGAATTTCA |
| 30 | GmMYB29_T03-R1 | AAAGAGGGAATTTCACA |
| 31 | GmMYB29_T04-L1 | TGGATTAACTATTTGAG |
| 32 | GmMYB29_T04-R1 | GGGAATTTCACAATTGA |
| 33 | GmMYB29_T04-R2 | TTCACAATTGAAGAAGA |
| 34 | GmMYB29_T05-L1 | TTTCACAATTGAAGAAG |
| 35 | GmMYB29_T05-R1 | TGACATGCTGGGGAACA |

In some embodiments, the TALENs comprise a TAL effector which binds to a sequence within a functional variant of the sequences disclosed herein, e.g. SEQ ID NO: 1-4 and 18. The term “functional variant of a nucleic acid or protein sequence” as used herein, for example with reference to SEQ ID NOs: 1-4 and 18, refers to a variant gene sequence or part of the gene sequence which retains the biological function of the non-variant gene. A functional variant also comprises a variant of the gene of interest encoding a polypeptide which has sequence alterations that do not affect function of the resulting protein, for example in non-conserved residues. Also encompassed is a variant that is substantially identical, i.e. has only some sequence variations, for example in non-conserved residues, to the unaltered, e.g., wild type, nucleic acid sequences of the genes as shown herein and is biologically active. As used herein, variants of a particular nucleotide or amino acid sequence according to the various aspects of the invention can have at least about 50% to at least about 99%, for example, at least about 75%, at least about 85%, at least about 86%, at least about 87%, at least about 88%, at least about 89%, at least about 90%, at least about 92%, at least about 94%, at least about 95%, at least about 96%, at least about 97%, at least about 98%, or at least about 99% or more sequence identity to that particular non-variant nucleotide sequence of the genes disclosed herein, such as one of SEQ ID NO: 1-4 and 18.

Sequence identity can be determined using sequence alignment programs which are well known in the art.

TALEN gene editing is based on the gene-specific targeting of two half-TALEN proteins containing DNA recognition domains and Fokl endonuclease domains encoded in the plasmid including an appropriate promoter, terminator, and other noncoding sequences necessary for the expression in the recipient plant cells. Fokl is only active in a dimeric combination, and two half TALENs are required to enable the endonuclease DNA cleavage necessary for TALEN gene editing. Introduction of TALENs into cells, for example, cells of soybean Glycine max, can be accomplished in multiple ways known to those skilled in the art. Common methods of introduction of gene-editing TALENs include transfection with Agrobacterium spp. carrying a plasmid including TALEN gene cassettes, biolistic bombardment with plasmids or mRNA encoding TALEN sequence cassettes, PEG-mediated transformation with plasmids or mRNA encoding TALEN sequence cassettes, and introduction of TALEN proteins by multiple methods including PEG-mediated transfection.

The methods provided herein therefore can be used to produce plant parts (e.g., seeds) or plant products (e.g., meal, oil) having decreased isoflavone content, as compared corresponding plant parts or products from the unaltered plants. The invention is further described in the following examples, which do not limit the scope of the invention described in the claims.

In some embodiments, regeneration of soybean plants with endonuclease-induced mutations in IFS1 and IFS2 genes can be accomplished as follows. In some embodiments, IFS1 and IFS2 genes can be knocked out by delivering one TALEN (pair) designed to target a sequence within both IFS1 and IFS2. To accomplish this, the IFS1 and IFS2 TALEN is cloned into a suitable DNA vector. Such vectors can be delivered to plant cells by Agrobacterium-mediated transformation or by using biolistics. Transgenic soybean plants expressing the TAL effector endonucleases can be generated using standard transformation protocols. The plants are transferred to soil, and after approximately 4 weeks of growth, a small leaf is harvested from each plant for DNA extraction and genotyping. Each DNA sample is first screened using PCR for the presence of T-DNA. Further PCR-based characterization of the IFS1 and IFS2 genes is used to assess whether mutations occurred.

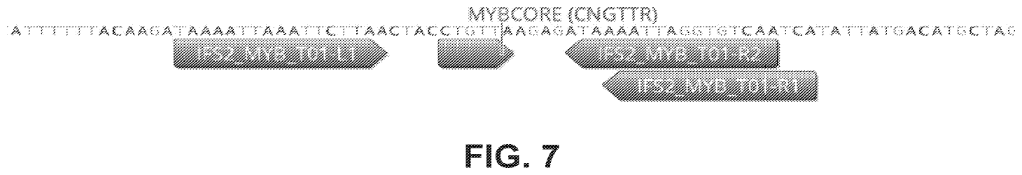

In some embodiments, regeneration of soybean plants with endonuclease-mediated deletion of the MYBCORE element in the IFS1 or IFS2 promoter can be accomplished as follows. Sequence analysis of the upstream region of IFS1 and IFS2 is performed to identify MYBCORE elements with the sequence CNGTTR, where N is any nucleotide and R is a purine (G or A). For IFS1 promoter, for example, four MYBCORE motifs are identified and TALEN pairs designed (FIG. 5). For IFS2 promoter, for example, one MYBCORE motif is identified and TALEN pairs designed (FIG. 7). TALEN pair(s) targeting the IFS1 promoter and IFS2 promoter, or both promoters, is/are cloned into a suitable DNA vector, delivered to a plant cell which is regenerated into a transgenic plantlet. After approximately 4 weeks of growth in soil, a small leaf is harvested from each plant for DNA extraction and genotyping. Each DNA sample is first screened using PCR for the presence of T-DNA. Further PCR-based characterization of the promoter region of the IFS1 and IFS2 genes is used to assess whether mutations occurred.

In some embodiments, regeneration of soybean plants with endonuclease-induced mutations in Myb29 can be accomplished as follows. The coding sequence of Myb29 is analyzed and TALEN are designed to induce a loss of function deletion that would prevent Myb29 from inducing IFS1 or IFS2 expression. TALEN pairs designed to bind to target sequences in exon 2 of Myb29 (e.g., within SEQ ID NO: 18) are delivered to soybean cells and regenerated to plants containing the desired mutations.

The invention will be further described in the following example, which does not limit the scope of the invention described in the claims.

### EXAMPLE

Experiments were conducted to create soybean plants with mutations in IFS1 and IFS2. To accomplish this, the GmlFS1_T01 TAL effector endonuclease pair (SEQ ID NO: 5 and 6 (Table 1)) was cloned into a bacterial vector, with TAL effector endonuclease expression driven by the nopaline synthase (NOS) promoter and delivered to soybean plant cells by Agrobacterium-mediated transformation or by using biolistics.

Transgenic soybean plants expressing the TAL effector endonucleases were generated using standard transformation protocols. Following transformation of wildtype soybean half cotyledons with sequences encoding the TAL effector endonuclease pair, putatively transgenic plants were regenerated. The plants were transferred to soil, and after approximately 4 weeks of growth, a small leaf was harvested from each plant for DNA extraction and genotyping. Multiple events were generated. All T₀ transgene-positive plants were then assayed to identify plants with mutations at the TAL effector endonuclease recognition sites.

The IFS1 and IFS2 PCR amplicons of positive plants were then inserted into cloning vectors and subjected to Sanger sequencing to confirm and characterize the mutant profiles. The resulting reads were then aligned to the wild type sequences to determine allele types.

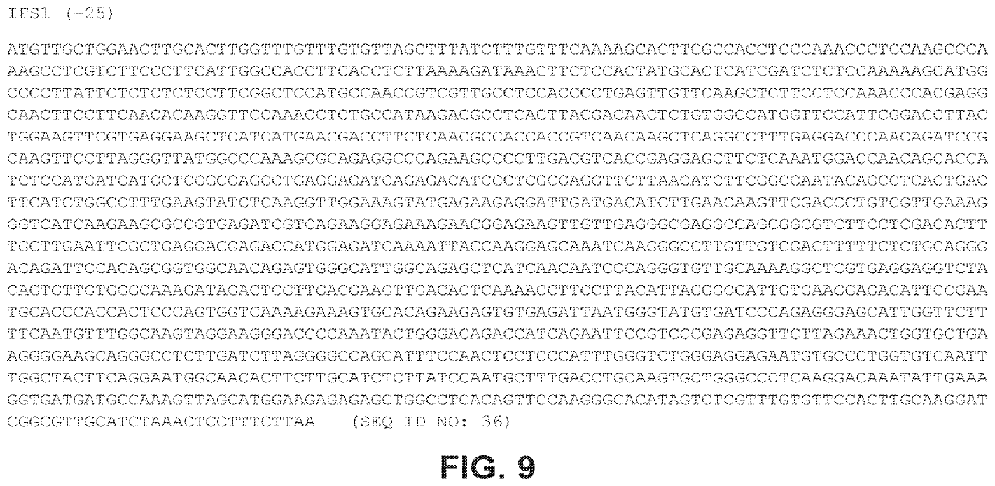

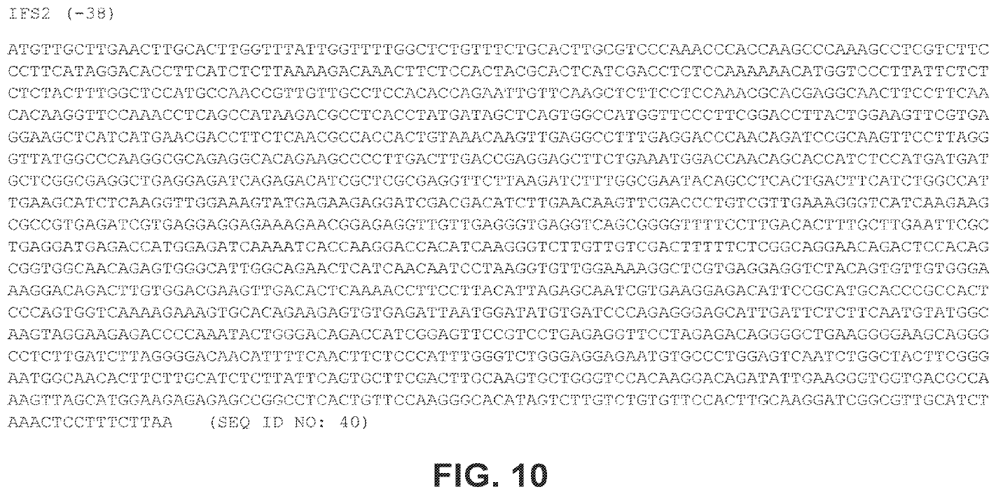

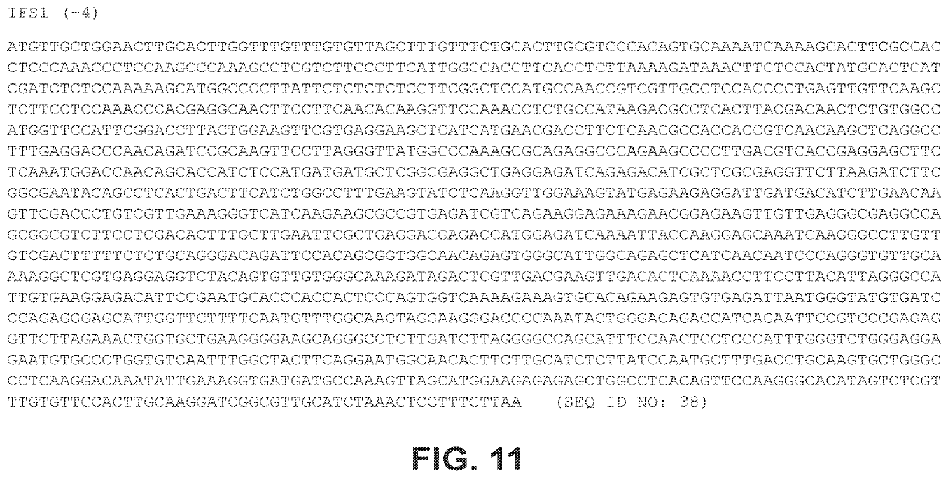

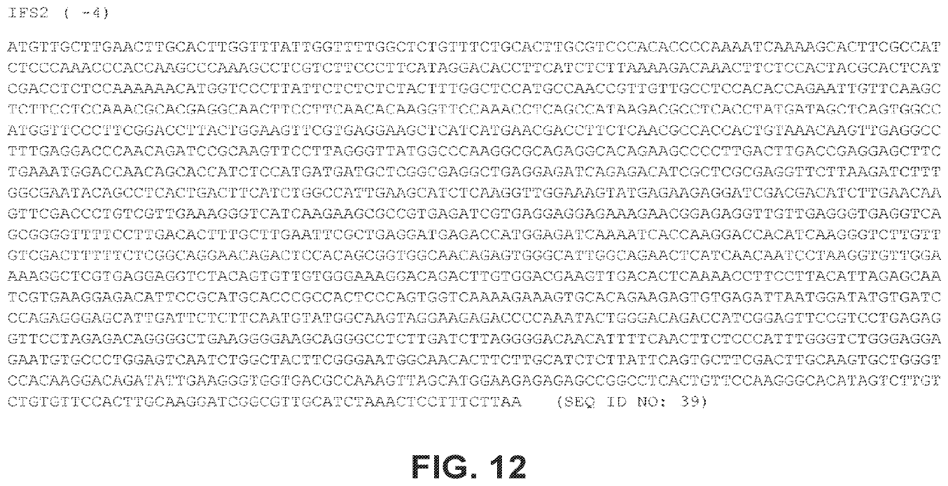

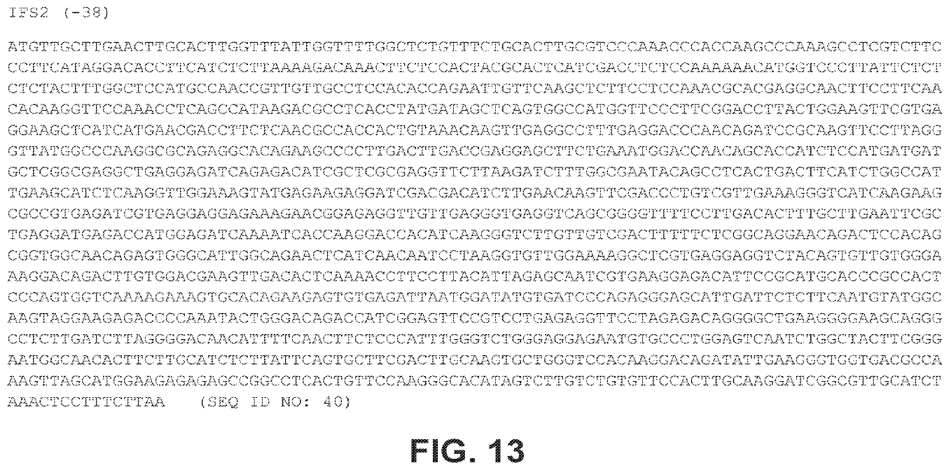

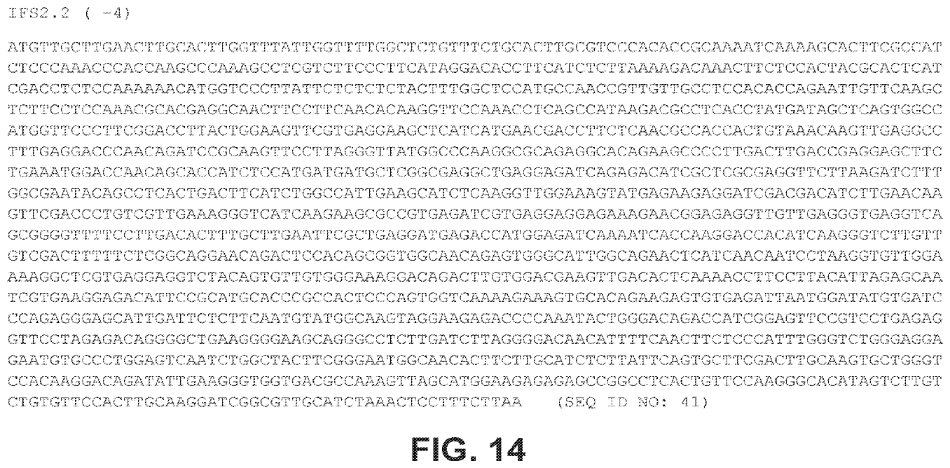

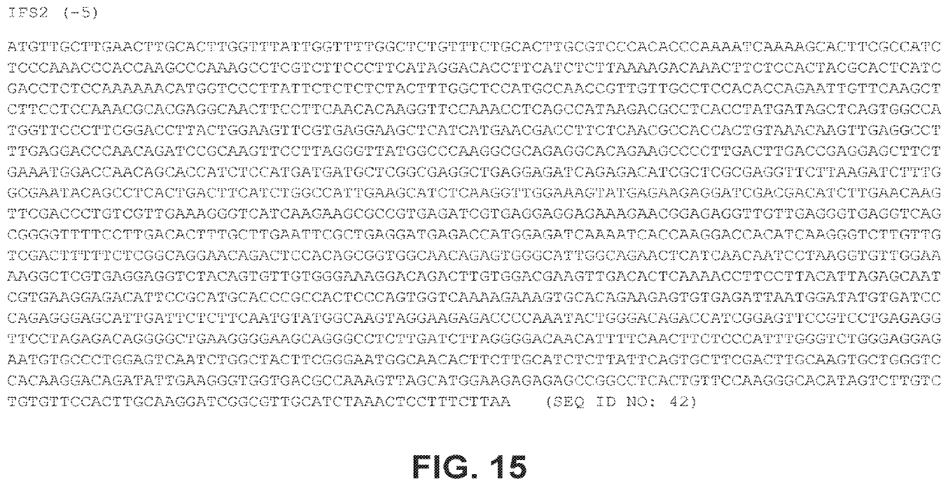

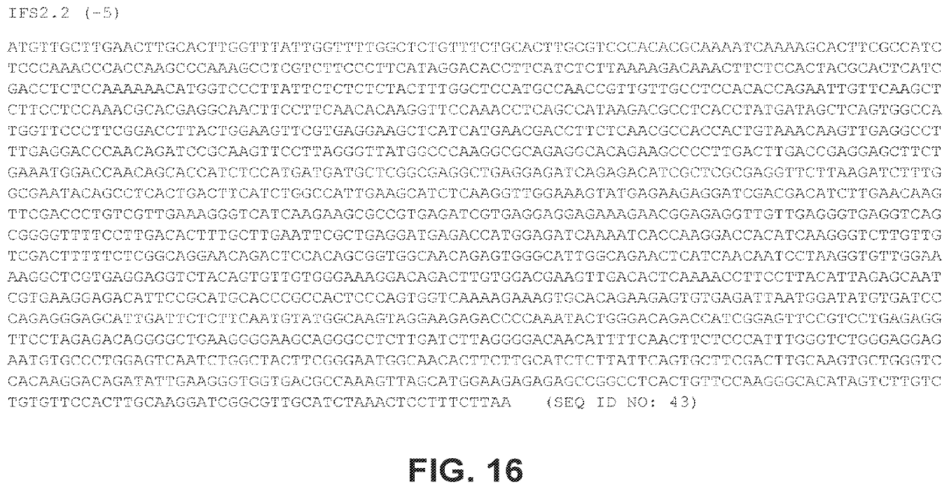

Representative sequences are shown in FIGS. 9-16 (SEQ ID NOs: 36-43).

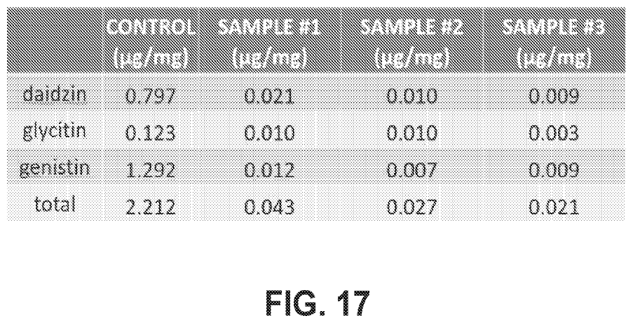

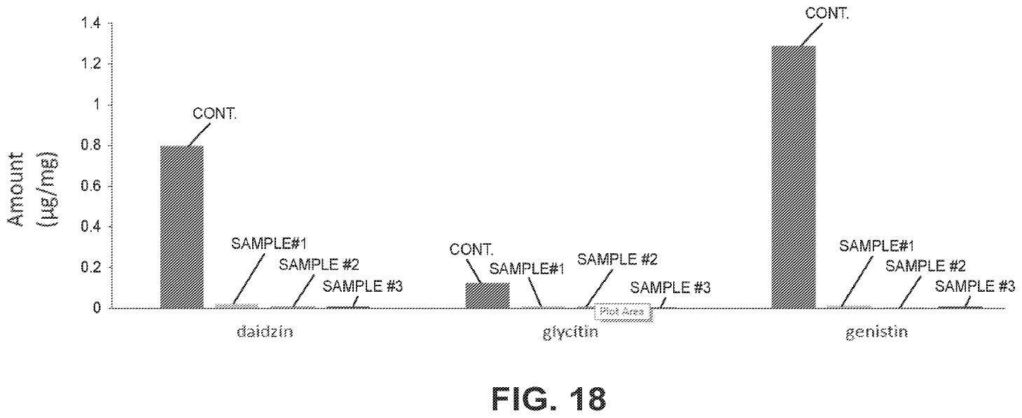

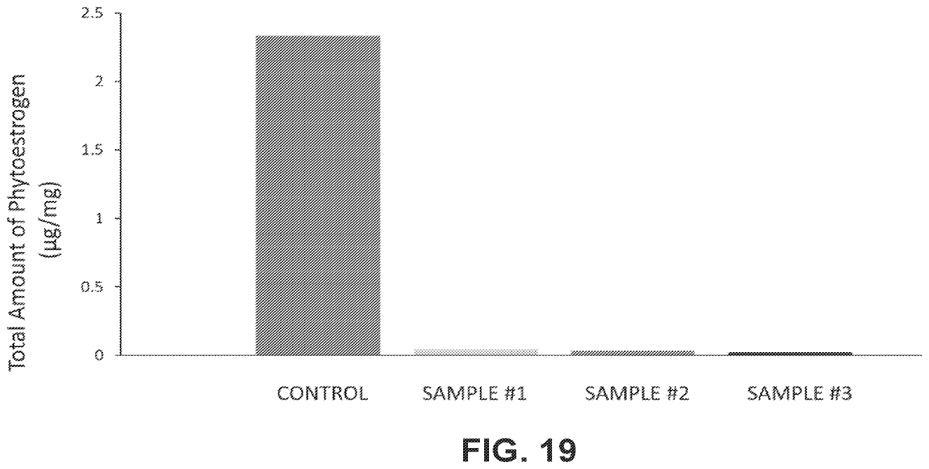

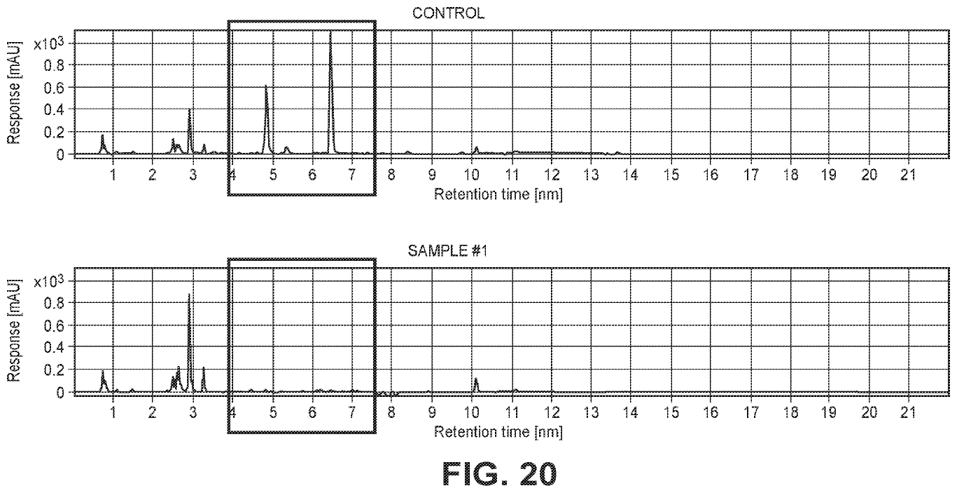

To determine the phytoestrogen profile on seeds produced from TAL effector endonuclease-induced mutations in Soybean IFS1 and IFS2 genes, soybean lines were grown to maturity and allowed to self-fertilize, giving rise to seeds that are homozygous mutant in IFS1 and IFS2 genes. These seeds were analyzed for phytoestrogen content by determining the total phytoestrogens content and the amount of daidzin, glycitin, and genistin. Briefly, individual soybean seeds from three mutant plant lines (#1-#3) and the wild-type plant line (Control) were pulverized with a mortar and pestle, and phytoestrogens were extracted from the pulverized tissue with 80% methanol. The extracts from the mutant plant lines exhibited an intense yellow hue, whereas the wild-type seed extract had a cream tint (not shown). The extracts were centrifuged and the supernatant was filtered. The filtered samples were hydrolyzed to determine non-esterified phytoestrogen forms using quantitative HPLC analysis (260 nm as the standard absorption wavelength). Representative chromatograms are shown in FIG. 20. Compared with the control seed, the results of the analysis show each mutant line produced seed with a significant reduction total phytoestrogen (55-100—fold change in total phytoestrogens (FIGS. 17 and 19)) and in each of daidzin, glycitin and genistin (FIGS. 17 and 18). For example, sample #3 seed exhibited a 98.87% reduction in daidzin, a 97.56% reduction in glycitin, and a 99.30% reduction in genistin compared with the wild-type seed (FIG. 17). Total phytoestrogens were reduced by 98.25% in Sample #1, 98.57% in Sample #2 and 99.07% in Sample #3 (FIG. 19).

As used herein, the term “about” indicates that the associated value can be modified, unless otherwise indicated, by plus or minus five percent (+/−5%) and remain within the scope of the embodiments disclosed.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise indicated, nucleic acids or oligonucleotides are written left to right in 5′ to 3′ orientation.

The words “herein,” “above,” and “below,” and words of similar import, when used in this application, refer to this application as a whole and not to any particular portions of the application, unless indicated otherwise. As used herein, the words “about” and “approximately” include minor variation around the stated value, usually within a standard margin of error, such as within 10% of the stated value.

The referenced patents, patent applications, and scientific literature referred to herein are hereby incorporated by reference in their entirety as if each individual publication, patent or patent application were specifically and individually indicated to be incorporated by reference.

While the preferred embodiment of the invention has been illustrated and described, it will be appreciated that various changes can be made therein without departing from the spirit and scope of the invention.

1. A soybean plant, plant part, or plant cell having reduced isoflavone content compared to an unaltered soybean plant, plant part, or plant cell, wherein the soybean plant, plant part, or plant cell comprises one or more mutations in:
the coding region of one or both IFS1 and IFS2 genes,
the coding region of a Myb29 gene,
the MYBCORE binding motif of the IFS2 promoter, or
the MYBCORE binding motif of the IFS1 promoter,
and wherein the plant, plant part, or plant cell is characterized by reduced expression of the one or both IFS1 and IFS2 genes compared with IFS1 or IFS2 gene expression in the unaltered soybean plant.

2. The soybean plant, plant part, or plant cell of claim 1, wherein the soybean plant, plant part, or plant cell comprises one or more mutations in the coding region of mutation in the coding region of the Myb29 gene.

3. The soybean plant, plant part, or plant cell of claim 1, wherein the soybean plant, plant part, or plant cell comprises one or more mutations in the coding region of the MYBCORE binding motif of the IFS1 promoter or the IFS2 promoter.

4. The soybean plant, plant part, or plant cell of claim 1, wherein the one or more mutations is introduced using a targeted genome modification using a rare-cutting endonuclease.

5. The soybean plant, plant part, or plant cell of claim 4, wherein the rare-cutting endonuclease is a transcription activator-like effector nuclease (TALEN).

6. The soybean plant, plant part, or plant cell of claim 1, wherein the mutation is introduced using a TALEN comprising a TAL effector which binds to a sequence within SEQ ID NO: 1 or SEQ ID NO: 2, or SEQ ID NO: 3 or SEQ ID NO: 4, or SEQ ID NO: 18.

7-8. (canceled)

9. The soybean plant, plant part, or plant cell of claim 1, wherein the mutation is introduced using a TALEN comprising a TAL effector which binds to one or more of SEQ ID NO: 5-17 or 19-35.

10. The soybean plant, plant part, or plant cell of claim 1, wherein the plant, plant part, or plant cell does not comprise a transgene.

11. The soybean plant, plant part, or plant cell of claim 1, wherein the plant part is seed.

12. The soybean plant, plant part, or plant cell of claim 1, wherein the plant, plant part, or plant cell has a content of isoflavones that is reduced by about 10%, about 20%, about 30%, about 50%, about 60%, about 70%, about 80%, or about 90% compared to the content of isoflavones in the unaltered soybean plant, plant part, or plant cell.

13. A method for producing a soybean plant, plant part, or plant cell with decreased content of isoflavones compared to an unaltered plant, plant part, or plant cell using targeted genome modification comprising introducing one or more mutations in the coding region of a Myb29 gene, the MYBCORE binding motif of the IFS2 promoter, the MYBCORE binding motif of the IFS1 promoter, the coding region of the IFS2 gene, the coding region of the IFS1 gene, or a combination thereof of the soybean plant, plant part or plant cell.

14. The method of claim 13, wherein the one or more mutations is introduced using a rare-cutting endonuclease.

15. The method of claim 14, wherein the rare-cutting endonuclease is a TALEN, optionally wherein the one or more mutations is introduced using a TALEN comprising a TAL effector which binds to a sequence within SEQ ID NO: 1 or SEQ ID NO: 2, SEQ ID NO: 3 or 4, or SEQ ID NO: 18.

16-18. (canceled)

19. The method of claim 13, wherein the one or more mutations is introduced using a TALEN comprising a TAL effector which binds to one or more of SEQ ID NO: 5-17 and 19-35.

20. The method of claim 13, wherein the soybean plant, plant part, or plant cell with decreased content of isoflavones does not comprise a transgene.

21. The soybean plant, plant part, or plant cell of claim 1, wherein the soybean plant, plant part, or plant cell comprises one or more mutations in the coding region of one or both IFS1 and IFS2 genes.

22. The soybean plant, plant part, or plant cell of claim 21, wherein the one or more mutations are present in the coding region of the IFS1 gene and the IFS2 gene.

23. The soybean plant, plant part, or plant cell of claim 22, wherein the mutation has been introduced using a TALEN comprising a first TAL effector that binds to SEQ ID NO: 5 and a second TAL effector that binds to SEQ ID NO: 6.

24. The method of claim 13, wherein the one or more mutations is introduced into the coding region of the IFS2 gene, the coding region of the IFS1 gene, or a combination thereof of the soybean plant, plant part or plant cell.

25. The method of claim 24, wherein mutations are introduced into the coding region of the IFS1 gene and the IFS2 gene, optionally wherein the one or more mutations are introduced using a TALEN comprising a first TAL effector that binds to SEQ ID NO: 5 and a second TAL effector that binds to SEQ ID NO: 6.
